(19) United States
(12) Patent Application Publication
(10) Pub. No.: US 2009/0125066 A1
(43) Pub. Date: May 14, 2009

(54) FACET STABILIZATION SCHEMES

(76) Inventors: Gary Kraus, Houston, TX (US); Jamal Taha, Cincinnati, OH (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 12/340,840

(22) Filed: Dec. 22, 2008

Related U.S. Application Data
(62) Division of application No. 11/351,270, filed on Feb. 9, 2006, now abandoned.
(60) Provisional application No. 60/651,163, filed on Feb. 9, 2005.
(60) Provisional application No. 60/678,954, filed on May 6, 2005.

(51) Int. Cl. A61B 17/70; A61F 2/44

(52) U.S. Cl. 606/279; 606/247; 623/17.11

(57) ABSTRACT

The present invention relates to methods of stabilizing a facet joint, wherein the facet joint comprises an inferior articular facet of the superior level, a superior articular facet of the inferior level, and an interfacet space that is defined between the opposing faces of the inferior and superior articular facets. These methods of stabilization may be achieved through a variety of applications and devices. In addition, the stabilization may be provided with various degrees of compression sufficient to stabilize the facet joint.

### CROSS-REFERENCE TO RELATED APPLICATIONS

This application is a division of U.S. patent application Ser. No. 11/351,270 filed Feb. 9, 2006 which claims the benefit of U.S. Provisional Patent Application Ser. Nos. 60/651,163, filed Feb. 9, 2005 (JAM 0006 MA), 60/678,954, filed May 6, 2005 (JAM 0007 MA).

### BRIEF SUMMARY OF THE INVENTION

The present invention relates to facet joint stabilization. According to the present invention, a variety of devices and methodologies are introduced as means for stabilizing one or more facet joints along the spinal column. One embodiment of the present invention relates to a method of stabilizing a facet joint through the insertion of one or more interfacet grafts into the interfacet space. This method generally comprises decorticating portions of the inferior and superior articular facets that define the interfacet space and inserting into those decorticated areas one or more interfacet grafts. These interfacet grafts are configured to encourage fusion of the facet joint.

Another embodiment of the present invention relates to a method of stabilizing a facet joint through the positioning of a compressible insert in the interfacet space. Thereby, the compressible insert is placed between the opposing faces of the inferior articular facet and the superior articular facet. This compressible insert generally comprises a degree of compressibility that is sufficient to support the interfacet space during movements of flexion and extension of the spine.

Another embodiment of the present invention relates to a method of stabilizing a facet joint through the insertion a fusion cage into the interfacet space. This fusion cage is configured to maintain a substantial degree of structural integrity when subject to flexion and extension in the facet joint. The provided fusion cage is inserted into the interfacet space such that the fusion cage engages both the inferior articular facet and the superior articular facet and, thereby, sufficiently stabilizes the facet joint.

Yet another embodiment of the present invention relates to a method of stabilizing a facet joint through the insertion of a fusion cage in a transfacet fashion. This method comprises removing corresponding portions of the inferior and superior articular facets so as to provide a cage accommodating channel in the inferior and superior articular facets and across the interfacet space. Thereafter, a fusion cage is provided and inserted into the channel such that the cage engages both the inferior articular facet and the superior articular facet and passes across the interfacet space.

Another embodiment of the present invention relates to a method of stabilizing a facet joint with the use of a cord peripherally wrapped around the facet joint. This method comprises forming cord accommodating notches in the cortical portions of the inferior and superior articular facets. A length of cord is then wrapped about portions of the inferior and superior articular facets such that the cord runs through the cord accommodating notches and about the facet joint with a degree of compression sufficient to stabilize the facet joint.

Yet another embodiment of the present invention relates to a method of stabilizing a facet joint through the threading and looping of a cord in a transfacet fashion. This method comprises removing corresponding portions of the inferior articular facet and the superior articular facet so as to provide a cord accommodating channel in the inferior articular facet, in the superior articular facet, and across the interfacet space. A length of a primary end of a cord is then thread through this channel. Thereafter, the primary end of the cord is secured to a secondary end of the cord so as to form a loop of the cord around at least a portion of the facet joint. This loop defines a degree of compression sufficient to stabilize said facet joint.

Another embodiment of the present invention relates to a method of stabilizing a facet joint through the threading of a cord in a transfacet fashion. This method comprises removing corresponding portions of the inferior and superior articular facets so as to provide a cord accommodating channel in the inferior articular facet, in the superior articular facet, and across the interfacet space. A length of a primary end of a cord is then threaded through this channel. At least one fixation device then binds the primary end and a secondary end of the cord to the interior articular facet and the superior articular facet with a degree of compression sufficient to stabilize the facet joint.

Yet another embodiment of the present invention relates to a method of stabilizing a facet joint through the use of facet stabilization hardware inserted through the interfacet space. This method comprises orienting a bone removal tool along a primary axis in the interfacet space substantially parallel to the opposing faces of the inferior and superior articular facets. Corresponding portions of the inferior articular facet and the superior articular facet are then removed by tilting the bone removal tool with respect to the primary axis about a pivot point along the primary axis, wherein the bone removal tool defines an operative removal surface extending at least partially beyond the bounds of the interfacet space to an extent sufficient to provide a hardware accommodating channel through the inferior articular facet, the superior articular facet, or both. The method further comprises positioning facet stabilization hardware in the hardware accommodating channel such that one or both ends of the hardware are exposed from cortices of the inferior articular facet and the superior articular facet. This facet stabilization hardware is configured to provide a degree of compression sufficient to stabilize the facet joint.

Accordingly, it is an object of the present invention to provide improved devices and methods for stabilizing facet joints. Other objects of the present invention will be apparent in light of the description of the invention embodied herein.

### BRIEF DESCRIPTION OF THE SEVERAL VIEWS OF THE DRAWINGS

The following detailed description of specific embodiments of the present invention can be best understood when read in conjunction with the following drawings, where like structure is indicated with like reference numerals and in which:

FIG. 1 is an illustration of an interfacet graft or a compressible insert positioned between the inferior articular facet of the superior level and the superior articular facet of the inferior level.

FIG. 2 is an illustration of an interfacet graft or a compressible insert and a cord wrapped about and through the facet joint and the interfacet graft or the compressible insert.

FIG. 3A is an illustration of a fusion cage inserted into the interfacet space and engaged with the opposing faces of the inferior articular facet of the superior level and the superior articular facet of the inferior level that define the interfacet space.

FIG. 3B is an illustration of a threaded exterior surface of a fusion cage.

FIG. 3C is a schematic, cross-sectional illustration of a fusion cage comprising an exterior surface and an interior space, both of which define a fastening configuration.

FIG. 3D is a schematic, cross-sectional illustration of a fixation device coupled to a fusion cage according to one embodiment of the present invention.

FIGS. 4A and 4B are illustrations of a fusion cage inserted into the facet joint in a transfacet fashion, wherein the ends of the fusion cage are engaged with fixation devices.

FIG. 5 is an illustration of a cord positioned in a cord accommodating notch and peripherally wrapped around the facet joint.

FIG. 6 is an illustration portraying a variety of facet stabilization schemes according to various embodiments of the present invention.

FIG. 7 is an illustration of a cord forming a loop configuration in a transfacet fashion with the use of a sleeve positioned around the cord.

FIG. 8 is an illustration of a cord forming a loop configuration in a transfacet fashion with the use of a buttress positioned inside the cord in relation to the facet joint.

FIGS. 9A and 9B are illustrations of a cord inserted through the interfacet space and positioned in a hardware accommodating channel.

### DETAILED DESCRIPTION

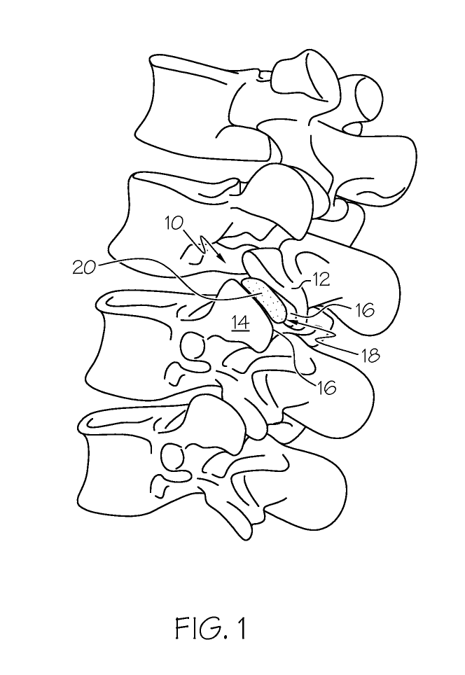

Referring initially to FIG. 1, the present invention relates to methods of stabilizing facet joints 10. For the purposes of defining and describing the present invention, it is noted that the facet joint 10 comprises an inferior articular facet 12 of the superior level, a superior articular facet 14 of the inferior level, and an interfacet space 18 that is defined between the opposing faces 16 of the inferior 12 and superior 14 articular facets.

One embodiment of the present invention, shown in FIG. 1, relates to a method of stabilizing a facet joint 10 through the insertion of at least one interfacet graft 20 into the interfacet space 18. This method comprises decorticating portions of the opposing faces 16 of the inferior 12 and superior 14 articular facets that define the interfacet space 18. For the purposes of describing and defining all of the methods of the present invention, it is noted that a facet decorticating process involves removing aspects of a facet portion of a vertebrae where such aspects include areas such as, but not limited to, the cortex and/or the synovium of a facet. The decorticating process may be, but is not necessarily, achieved through the use of a suitable conventional or yet to be developed osteotome device that is configured to remove aspects of a facet. For example, and not by way of limitation, an osteotome device may be positioned over the interfacet space 18 and embedded into the inferior articular facet 12 or the superior articular facet 14, or both. The osteotome device can then operated to remove consistent, pre-defined pieces of the inferior 12 and/or superior 14 articular facets, including portions of the opposing faces 16 of the inferior 12 and superior 14 articular facets that define the interfacet space 18.

Alternatively, the decorticating process may be, but is not necessarily, achieved through the use of a drill bit or similar decorticating hardware that is inserted into the interfacet space 18. In one embodiment, the drill bit comprises a center channel bored out allowing the drill bit to be positioned over a guide wire that is inserted into the interfacet space. The guide wire is configured such that it passes through the center channel of the drill bit as the drill bit is inserted into the interfacet space 18. This drill bit is thereby configured to operatively slide back and forth along the longitudinal axis of the guide wire such that the opposing faces 16 of the inferior 12 and superior 14 articular facets that define the interfacet space 18 are effectively decorticated.

The osteotome device or other decorticating device, may be guided, similarly to the drill bit described above, into proper position in relation to the interfacet space 18 by a guide wire, such as, but not limited to, a Kirschner wire, also called a K-wire—a sharp metal pin that can be used to hold bone fragments in place. This guide wire typically is inserted into the interfacet space 18 either through an open surgical procedure or under X-ray or fluoroscopic guidance.

Following the decorticating process, this method comprises positioning at least one interfacet graft 20 in the interfacet space 18, such that the interfacet graft 20 is in biological communication with the decorticated portions of the opposing faces 16 of the inferior 12 and superior 14 articular facets. This interfacet graft 20 is configured to encourage fusion of the opposing faces 16 of the inferior 12 and superior 14 articular facets. The fusion that results from this method provides the immobilization and the stabilization of the facet joint 10. For the purposes of describing and defining the present invention, it is understood that objects are considered to be in biological communication with each other if they are in a configuration where one of the objects can affect the biological condition of the other. For example, in the context of the interfacet graft, the presence of the graft in the interfacet space causes the growth of bone on each of the decorticated portions of the opposing faces of the inferior and superior articular facets.

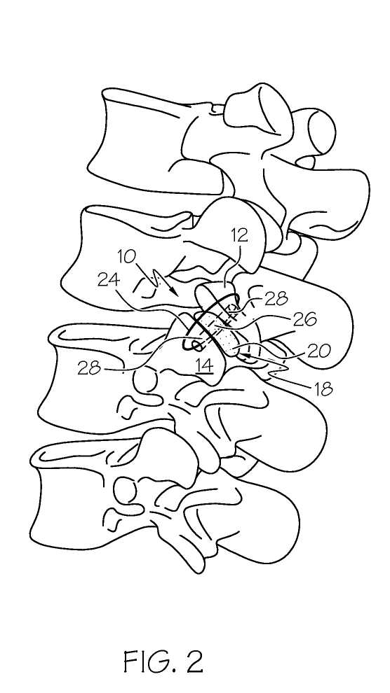

As shown in FIG. 2, this method may, but need not necessarily, comprise further stabilizing the facet joint 10 with facet stabilization hardware following the positioning of the interfacet graft 20 in the interfacet space 18. With the use of facet stabilization hardware, the interfacet graft 20 may be configured to function as a guide 26 for the facet stabilization hardware. In the illustrated embodiment, the facet stabilization hardware comprises a cord 24 configured to compress the facet joint 10 about the interfacet graft 20. More specifically, as is illustrated in FIG. 2, the interfacet graft 20 may be configured to guide the cord 24 in a configuration where the cord 24 passes through the guide portion 26 of the interfacet graft 20 positioned in the interfacet space 18 and through one or more channels 28 drilled, or otherwise provided, in the inferior 12 and superior 14 articular facets. Alternatively, the interfacet graft 20 may be sized to protrude slightly beyond the bounds of the interfacet space 18 and may be provided about its periphery with grooves, slots, channels or other guiding elements configured to guide a cord 24 that is at least partially wrapped about the facet joint 10. It is further contemplated that the interfacet graft 20 may be replaced with hardware that is primarily intended to guide the facet stabilization hardware and is not targeted for enhancing bone growth. In which case the hardware replacing the interfacet graft may merely be formed of a metal, metal alloy, or other suitable surgical grade materials.

The interfacet graft 20 described above may be configured in an exact shape and size that corresponds with the consistent, pre-defined pieces of the opposing faces 16 of the inferior 12 and superior 14 articular facets that define the interfacet space 18 removed by the osteotome device. Alternatively, the interfacet graft 20 may be configured in any shape suitable for this method. In addition, the interfacet graft 20 may be perforated or non-perforated, threaded or non-threaded, with or without grooves, beveled or flat, hollow in the center or solid, etc.

Another embodiment of the present invention, also depicted in FIGS. 1 and 2, relates to a method of stabilizing a facet joint through the insertion of at least one compressible insert 20 into the interfacet space 18. This method comprises positioning at least one compressible insert 20 in the interfacet space 18 such that the compressible insert 20 is placed between the opposing faces 16 of the inferior 12 and superior 14 articular facets. This compressible insert 20 generally comprises a degree of compressibility that is sufficient to support the interfacet space 18 during movements of flexion and extension of the spine. The compressible insert 20 may be configured with various degrees of compressibility. The flexion and extension of the spine is dependant upon the degree of compressibility of the compressible insert 20 positioned in the interfacet space 18. In addition, the compressible insert 20 may be configured in any shape or size suitable for this method and may be configured to function as a guide for facet stabilization hardware. This method may, but need not necessarily, further comprise decorticating portions of the opposing faces 16 of the inferior 12 and superior 14 articular facets such that a compressible insert 20 is thereafter placed between the decorticated portions of the opposing faces 16 of the inferior 12 and superior 14 articular facets that define the interfacet space 18.

Both the interfacet graft 20 and the compressible insert 20 may be provided with diagnostically-opaque features to enable the convenient monitoring of the structural integrity of the interfacet graft 20 and the compressible insert 20. For purposes of the present invention, the term “diagnostically-opaque” means that the interfacet graft 20 and the compressible insert 20, and all hereinafter described components utilized in stabilizing a facet joint, may be diagnostically recognized through advanced diagnostic imaging procedures, such as, but not limited to, X-ray, CAT-scan, fluoroscopy, magnetic resonance imaging (MRI). With the components utilized in stabilizing a facet joint being internally implanted into the patient, there is a need for diagnostic recognition of these components to ensure their structural integrity without the need for a surgical procedure. Therefore, the diagnostically-opaque features allow for the periodic non-invasive monitoring of the structural integrity of the components utilized in stabilizing a facet joint. It is contemplated that the interfacet graft 20 and the compressible insert 20, and/or other components utilized in stabilizing a facet joint 10, may be provided with diagnostically-opaque features in a variety of ways. For example, and not by way of limitation, diagnostically-opaque material may be provided in discrete elements, or dispersed within the material forming the interfacet graft 20 and the compressible insert 20, or other components, or provided in any other manner that would permit the structural state of the interfacet graft 20 and the compressible insert 20, or other components, to be monitored periodically over time. In addition, specific teachings regarding the material forming interfacet grafts 20 and the compressible inserts 20 may be gleaned from existing or yet to be developed technology.

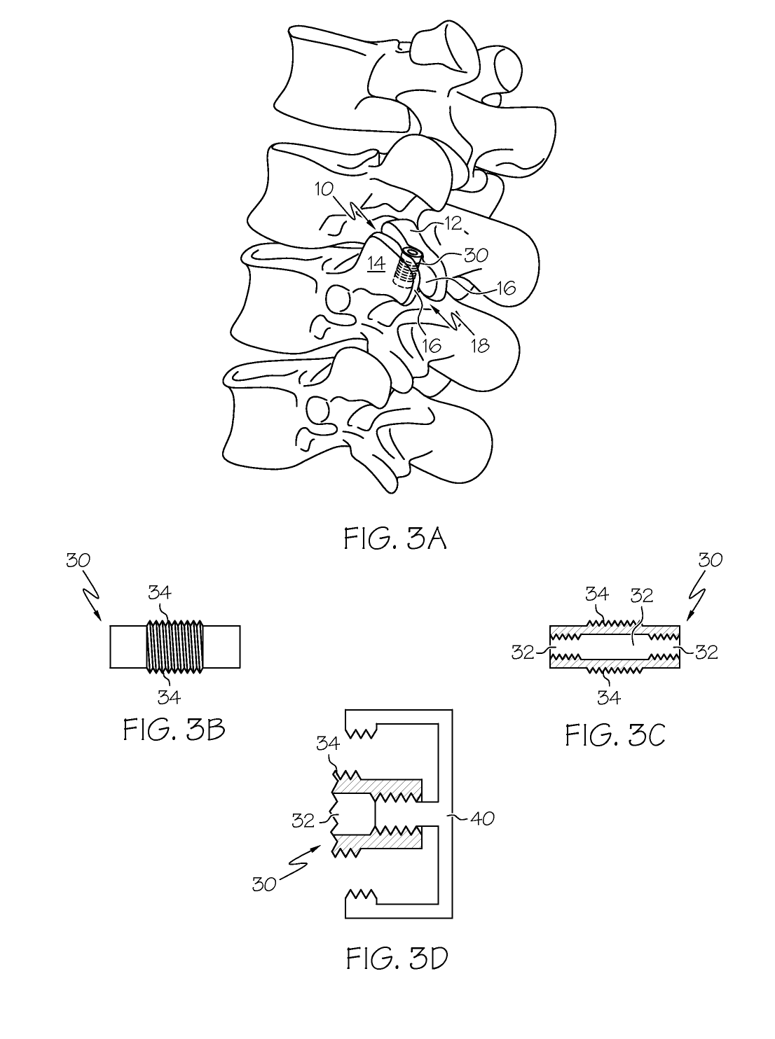

Another embodiment of the present invention, shown in FIG. 3A, relates to a method of stabilizing a facet joint 10 through the insertion of a fusion cage 30 into the interfacet space 18. This method generally, but not necessarily, comprises removing corresponding portions of the inferior articular facet 12 and the superior articular facet 14 that define the interfacet space 18 so as to provide an expanded interfacet space. It is contemplated by the present invention that the stabilization of a facet joint 10 through the insertion of a fusion cage 30 into the interfacet space 18 may be achieved without providing an expanded interfacet space 18. The particular process of expanding the interfacet space 18 will vary depending on the type of fusion cage 30, hereinafter described, that is inserted into the interfacet space 18. For example, and not by way of limitation, a guide wire may be positioned in the interfacet space 18 to serve as a localizing device for a variety of mechanisms that may be used to expand the interfacet space 18. For example, and not by way of limitation, the expanding process may be achieved by a distraction plug placed into the interfacet space 18 that distracts the interfacet space 18 open. A tang retractor may also be utilized in conjunction with a distraction plug or independently. This tang retractor may be used with an expandable tubular guide that enables further access to the facet joint 10. A drill bit, or other similar device, with or without the use of a distracter or retractor, is inserted into the interfacet space 18 to bore out portions of the opposing faces 16 of the inferior 12 and superior 14 articular facets, thereby expanding the interfacet space 18. A tap may be inserted into the interfacet space 18 to carve a groove into the opposing faces 16 of the inferior 12 and superior 14 articular facets.

This method generally comprises providing a fusion cage 30 that generally is configured to maintain a substantial degree of structural integrity when subject to flexion and extension in the facet joint 10. This fusion cage 30 is then inserted into the interfacet space 18 such that the fusion cage 30 engages both the inferior articular facet 12 and the superior articular facet 14. The insertion of the fusion cage 30 may be performed through either an open or a closed, minimally invasive, surgical procedure.

For the purposes of describing and defining all of the methods of the present invention, it is noted that a fusion cage 30 is a device that aids in the merging into a union, or unified structure, the inferior 12 and superior 14 articular facets either through mechanical engagement or through biological grafting. The cross-section of the fusion cage 30 may be configured in a variety of shapes, such as, but not limited to, cylindrical or block. Additionally, the fusion cage 30 may be solid, such as when comprised of bone material, or it may define an open interior space 32, as depicted in FIG. 3C, with a porous or non-porous exterior. The cage may be made of metal or metal alloys, carbon fiber, synthetic materials, or any other material suitable for surgical implantation within a patient.

When the fusion cage 30 is provided in a porous configuration, it can be configured with a degree of porosity sufficient to permit diffusion of biological substances into or from an interior space 32 of said fusion cage 30. These biological substances typically comprise bone or bone growth enhancing materials. Therefore, when the fusion cage 30 is provided in a porous configuration, the method of stabilizing the facet joint 10 further comprises at least partially filling the interior space 32 of the cage 30 with bone or bone growth enhancing materials to encourage fusion of the facet joint 10.

The fusion cage 30 may be fabricated from a variety of suitable materials including, but not limited to, surgical grade stainless steel, titanium, other metals or metal alloys, synthetic materials, carbon, graphite, combinations thereof, or any other suitable surgical material. In addition, specific teachings regarding the material forming the fusion cage 30 may be gleaned from existing or yet to be developed technology related to fusion cages 30.

Furthermore, as shown in FIGS. 3B and 3C, the exterior surface 34 of the fusion cage 30 may be provided in a fastening configuration so as to securely engage with the inferior articular facet 12 and the superior articular facet 14. For example, but not by way of limitation, the exterior surface 34 of the fusion cage 30 may be threaded, grooved, notched, or otherwise configured to engage securely with the inferior 12 and superior 14 articular facets. Alternatively, the exterior surface 34 may be smooth and not in a fastening configuration.

Referring to FIGS. 3C and 3D, the fusion cage 30 can also be designed to enable one or more fixation devices 40, hereinafter described, to engage the fusion cage 30. For example, and not by way of limitation, an interior space 32 of the fusion cage 30 can be provided in a fastening configuration. As depicted in FIG. 3D, a fixation device 40 can be configured to correspond with the fastening configuration of the interior space 32 of the fusion cage 30 such that the fixation device 40 may engage the interior space 32. The fixation device 40 can also be configured to compress the facet joint 10 as the fixation device 40 engages the interior space 32 of the fusion cage 30. Furthermore, this secure positioning of the fixation device 40 about the facet joint 10 may be achieved through the clamping, wrapping, or otherwise tightening of the fixation device 40 around the outside of the facet joint 10, serving to compress the inferior 12 and superior 14 articular facets around the fusion cage 30, or to prevent the fusion cage 30 from migrating out of the interfacet space 18. For example, and not by way of limitation, the fixation device 40 may be a washer/nut assembly, a bracket, a clamp, a washer/crimp assembly, or a binding sleeve. The fixation device 40 may also be provided in a contoured, conforming, compressible, smooth, roughened, and/or toothed surface and/or with a broad surface that widely distributes compressive forces across the facet joint 10 and inhibits movement of the fixation device 40 once it is engaged with the fusion cage 30 or other stabilization device. As an alternative to threads, grooves, notches, or other similar configurations, a snap or ratchet design may be utilized in enabling a fixation device 40 to engage the fusion cage 30 securely.

It is contemplated that all of the methods of the present invention described herein may further comprise stabilizing a facet joint 10 by coupling a facet joint 10 to one or more contiguous or non-contiguous facet joints 10. This coupling may be achieved with the use of one or more coupling mechanisms, such as, but not limited to, rods, wires, plates, bands, cords, or other similar mechanisms, or combination thereof. These coupling mechanisms may be configured with various degrees of flexibility. The amount of stabilization provided to said coupled facet joints is dependant upon the degrees of flexibility of the coupling mechanisms thereby coupled. Typically, but not necessarily, the fixation device 40 is configured to function as a securing receptacle for the coupling mechanisms. The securing receptacle may be fixed or have the ability to rotate. By way of example, and not of limitation, a nut/bolt/washer assembly version of a fixation device 40 could be configured to receive and secure a spinal rod. Similar provisions could be made in the facet loop/crimp, facet cord mechanisms, and other fixation devices 40 so as to receive and secure other types of coupling mechanisms. Alternatively, a securing receptacle may be attached to the coupling mechanism, which may then attach to the fixation device 40.

It is further contemplated that coupling mechanisms can be utilized in accordance with the present invention, along opposite lateral sides of the spinal column. These coupling mechanisms can also be cross-linked to each other utilizing cross-linking techniques similar to those employed in cross-linking pedicle screw/rod constructs.

It is also contemplated by the present invention that the coupling mechanisms may be secured to facet joints 10 by methods other than securing the coupling mechanisms and fixation devices 40 together. Additionally, it is further contemplated that the coupling mechanisms may be secured to aspects of the spine other than, or in addition to, a facet joint, such as, but not limited to, a pedicle, a spinous process, a transverse process, or any other aspect of the spine, or a combination thereof.

Further, the cross-section of the coupling mechanisms may be circular, multi-sided, band-like, or contoured, or other similar configuration. Coupling mechanisms according to the present invention may be fabricated from a variety of suitable materials including, but not limited to, surgical grade stainless steel, titanium, other metals or metal alloys, synthetic materials, carbon, graphite, combinations thereof, or any other suitable surgical material. In addition, specific teachings regarding the material forming the coupling mechanisms may be gleaned from existing or yet to be developed technology related to coupling mechanisms.

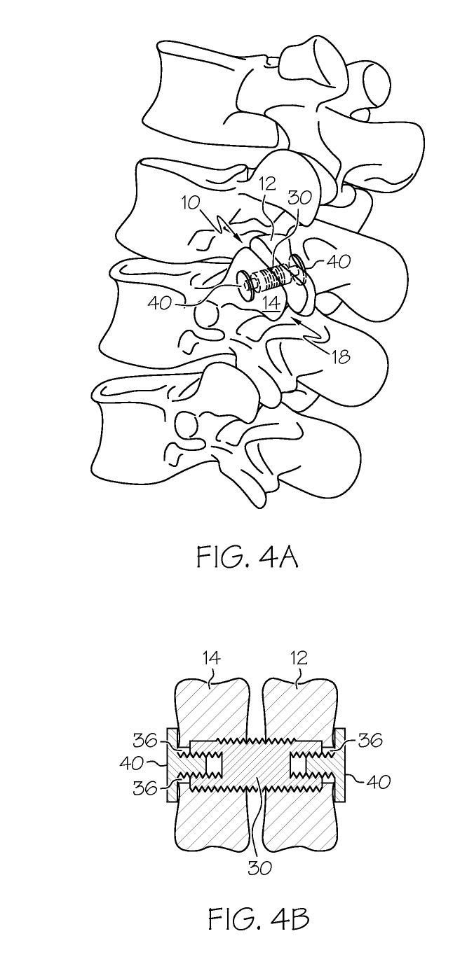

Another embodiment of the present invention, shown in FIGS. 4A and 4B, relates to a method of stabilizing a facet joint 10 through the insertion of a fusion cage 30 in a transfacet fashion. For the purposes of describing and defining the present invention, it is noted that the term “transfacet” refers to a configuration where particular hardware or channels cross into or through the inferior articular facet 12 and the superior articular facet 14, across the interfacet space 18. The method illustrated in FIGS. 4A and 4B comprises removing corresponding portions of the inferior articular facet 12 and the superior articular facet 14 so as to provide a cage accommodating channel 36 in the inferior articular facet 12, in the superior articular facet 14, and across the interfacet space 18.

The cage accommodating channel 36 may be provided in a variety of ways. For example, and not by way of limitation, a drill bit, or other similar device, may be utilized to bore a hole through the inferior articular facet 12, across the interfacet space 18, and into the superior articular facet 14. It is contemplated that the drill bit may just as easily first bore a hole through the superior articular facet 14 and then into the inferior articular facet 12. It is further contemplated by this method that the hole bored into whichever articular facet is secondly penetrated by the drill bit may end within, or pass entirely through, that articular facet.

This method further comprises providing a fusion cage 30 and inserting this fusion cage 30 into the cage accommodating channel 36 such that the fusion cage 30 engages both the inferior articular facet 12 and the superior articular facet 14 and passes across the interfacet space 18. This is accurately depicted in FIG. 4B. The fusion cage 30 generally is then bound to the inferior 12 and superior 14 articular facets by at least one fixation device 40 with a degree of compression sufficient to stabilize the facet joint 10.

The fixation device 40 provided with this method typically is configured as a washer/nut assembly, or other similar device or assembly, wherein the fixation device 40, or an aspect of the fixation device 40, comprises a broad surface. The broad surface may be configured in a variety of shapes, sizes, and contours so as to proportionally fit variously sized facets. This broad surface is positioned about a portion, or portions, of the facet joint 10 and thereby dispenses the compressive force over a wide area of the inferior articular facet 12 and/or superior articular facet 14 as the fixation device 40 secures to the fusion cage 30. The degree of compression provided by the broad surface of the fixation device 40 allows for the immediate stabilization of the facet joint 10. This immediate stabilization may be particularly useful in stabilizing osteoporotic, or otherwise compromised, bone.

Alternatively, a fusion cage 30 provided in this method may be configured with a threaded leading edge. When introduced to an inferior articular facet 12 or a superior articular facet 14, this threaded leading edge engages and inserts into the selected facet while a remaining smooth, lagging part allows a bolt or a washer to pull the facet into compression. The threaded leading edge is inserted until it crosses the interfacet space 18 and penetrates and engages the secondary facet. It is contemplated that this lagging concept may be applied to a screw or bolt as well, wherein the lagging part may be solid or hollow.

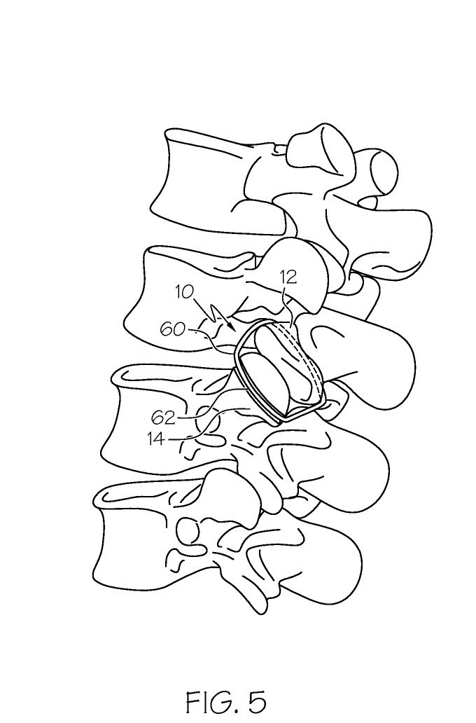

Yet another embodiment of the present invention, shown in FIG. 5, relates to a method of stabilizing a facet joint 10 with the use of a cord 60 peripherally wrapped around a facet joint 10. This method typically comprises forming cord accommodating notches 62 in cortical portions of the inferior 12 and superior 14 articular facets. Thereafter, the method generally comprises wrapping a length of cord 60 about portions of the inferior articular facet 12 and the superior articular facet 14 such that the cord 60 runs through the cord accommodating notches 62 and about the facet joint 10 with a degree of compression sufficient to stabilize the facet joint 10. It is contemplated by the present invention that this method may, but need not necessarily, further comprise removing corresponding portions of the inferior 12 and superior 14 articular facets so as to provide a cord accommodating channel in the inferior articular facet 12, in the superior articular facet 14, and across the interfacet space 18. The cord 60 may then be passed through the cord accommodating channel such that the cord 60 is both wrapped peripherally and passed in a transfacet fashion in relation to the facet joint 10, thereby providing additional stabilization to the facet joint 10.

The cord 60 of the present invention may be configured to function elastically or inelastically. For example, and not by way of limitation, the cord 60 may be a metal wire, a steel cable, a braided wire cable, a polymer cord, or other similar elastic or inelastic cord, cable, or band. It is contemplated by the present invention that this method may further comprise inserting into the interfacet space 18 a cord migration prevention device configured to engage portions of the length of the cord 60 wrapped about portions of the inferior 12 and superior 14 articular facets. Although the cord migration prevention device may take a variety of forms, it is noted that, according to one aspect of the present invention, the device may be sized to protrude slightly beyond the bounds of the interfacet space 18 and may be provided about its periphery with grooves, slots, channels or other guiding elements configured to guide a cord 60 that is wrapped about the facet joint 10. The cord migration prevention device may also be provided with one or more openings for permitting the passage of a transfacet cord, or other transfacet or interfacet hardware.

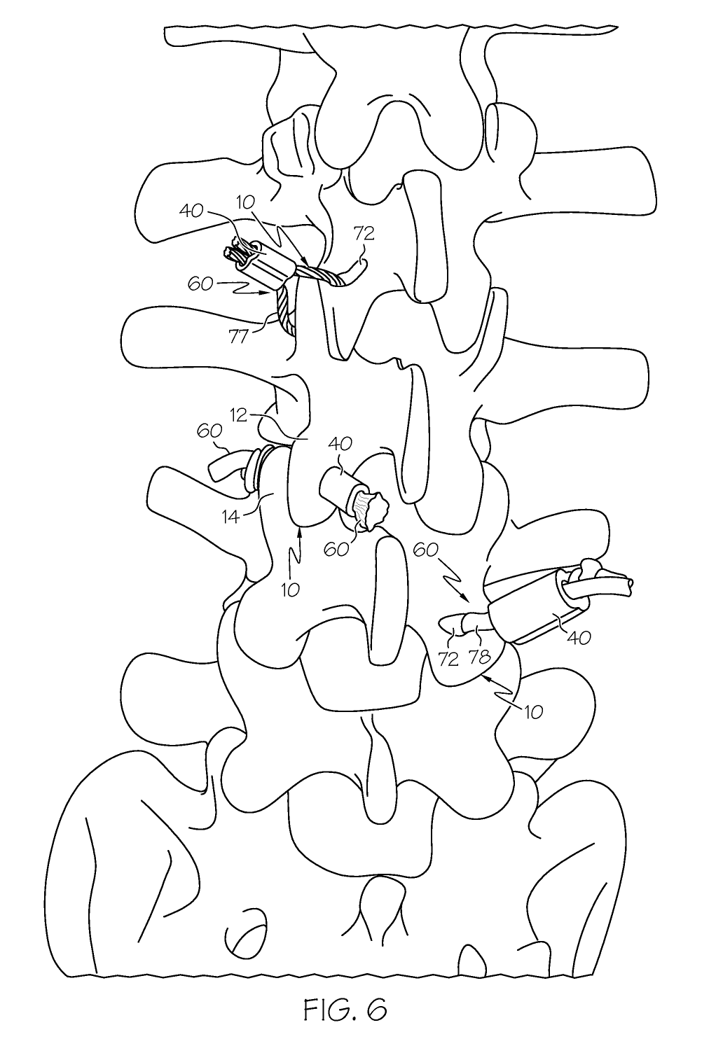

Another embodiment of the present invention, shown in different embodiments in FIGS. 6, 7, and 8, relates to a method of stabilizing a facet joint 10 through the threading and looping of a cord 60 in a transfacet fashion. In FIG. 6, one of the loops is made of braided wire 77 and the wire is only loosely crimped down, for illustrative purposes. In actual use, the loop would be tighter. Also in FIG. 6, a more flexible cord 78 is used to create another loop and, as such, is referred to herein as a dynamic facet loop because it permits a greater degree of movement in the facet joint. Likewise, the loop would be tightened around the facet.

This method generally comprises removing corresponding portions of the inferior articular facet 12 and the superior articular facet 14 so as to provide a cord accommodating channel 72 in the inferior articular facet 12, in the superior articular facet 14, and across the interfacet space 18. It is contemplated by the present invention that this cord accommodating channel 72 may be provided through either an open or a closed, minimally invasive, surgical procedure. Thereafter, a length of a primary end of a cord 60 is threaded through the channel 72. It is contemplated by the present invention that the primary end of the cord 60 may be attached to a leading edge point, a drill bit, or other bone removing device, thereby facilitating the threading of the cord 60 through the cord accommodating channel 72. Following the threading of the cord 60 through the channel, the primary end is secured to a secondary end of the cord 60 so as to form a loop of the cord 60 around at least a portion of the facet joint 10 with a degree of compression sufficient to stabilize the facet joint 10. The securing of the primary end and the secondary end is accomplished with the application of at least one fixation device 40 that permits the loop to tighten, but prevents the loop from loosening.

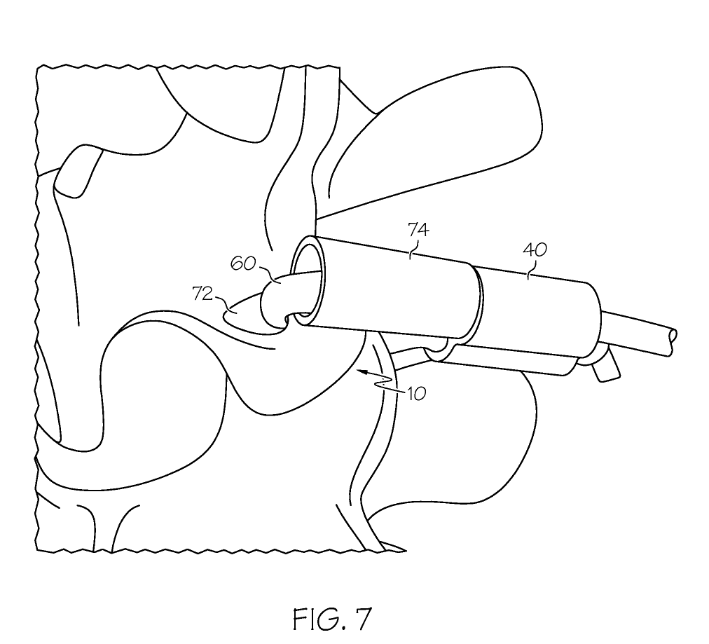

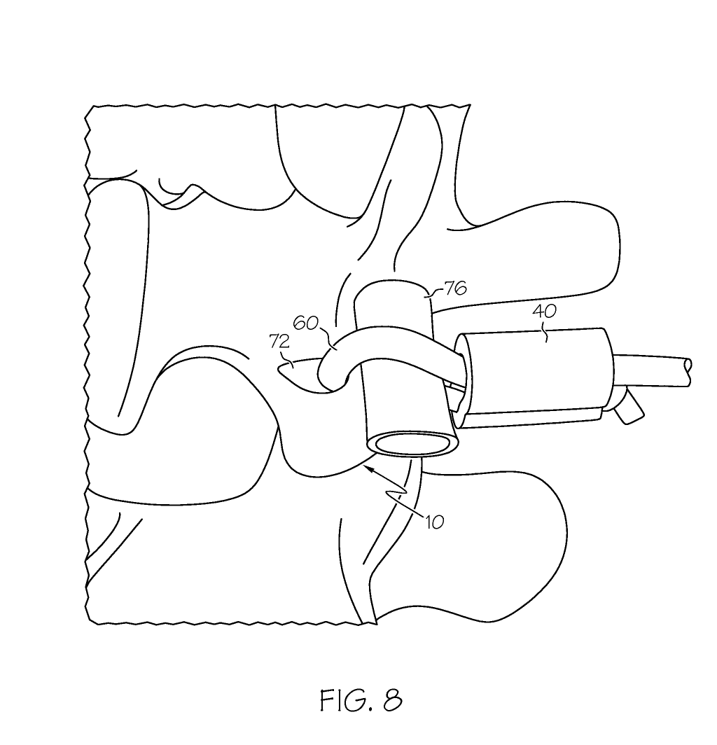

This method of the present invention may, but need not necessarily, further comprise positioning facet stabilization hardware between the cord 60 and the facet joint 10 so as to further stabilize the facet joint 10. It is noted by the present invention that the facet stabilization hardware may be positioned around a portion, or portions, of the cord forming the loop as a sleeve 74 (shown in FIG. 7) or other similar device, or may be positioned inside a portion, or portions, of the cord forming the loop in relation to the facet joint, as a buttress 76 (shown in FIG. 8) or other similar device. In either position, the facet stabilization hardware remains between the cord 60 and the facet joint 10. Such positioning of the facet stabilization hardware serves to reduce wear, degradation, and the risk of failure of the cord 60 forming the loop.

Typically, the facet stabilization hardware is configured as a sleeve 74 or a buttress 76, but other similar devices may be utilized as well. The sleeve 74 and the buttress 76 generally are configured to provide varying degrees of compression to the facet joint 10. Such characteristics of variable compressibility may be particularly useful in the context of dynamic stabilization, where it may be important to vary the degree to which a specific facet joint 10 is stabilized. The facet joint 10 is thereby further stabilized under this method by utilizing at least one sleeve 74 or at least one buttress 76, or both, to compress the facet joint 10. It is contemplated by the present invention, however, that the facet joint 10 may be sufficiently stabilized through the threading and looping of a cord 60 in a transfacet fashion either without the utilization of a sleeve 74 or buttress 76, or in conjunction with any combination or permutation thereof. It is further contemplated that the cord 60, the sleeve 74, and the buttress 76 may be configured with various degrees of elasticity and compressibility. The level of stabilization provided to the facet joint 10 is thereby dependant upon the degrees of elasticity and compressibility of the cord 60, the sleeve 74, and/or the buttress 76 positioned about or adjacent to the facet joint 10.

For example, where at least one sleeve 74 is positioned about portions of the cord 60, or loop, that contact the facet joint 10, the sleeve 74 may be configured such that it can be compressed along the length of those portions of the cord 60. In this manner, the extent to which the sleeve 74 is compressed and the compliance, compressibility, or rigidity of the material of which the sleeve 74 is comprised will directly correlate to the degree of rigidity imparted to the stabilization of the facet joint 10. Although the sleeve 74 may be secured in the compressed state in a variety of ways, it is noted that a fixation device 40 may be engaged about the free ends of the loop and secured over top of an end portion of the compressed sleeve 74. Alternatively, the fixation device 40 may actually surround and secure on a portion of the sleeve 74, either at the end or at another location on the sleeve 74. Additionally, if more than one sleeve 74 is positioned on each side of the loop about the facet joint 10, the fixation device 40 may be engaged over top of, or adjacent to, the end portions of the compressed sleeves 74.

It is contemplated that a compressible or non-compressible buttress 76 may be substituted for or used in addition to the various sleeves 74 of the present invention, particularly where it may be advantageous to prevent wear between a portion of the facet joint 10 and a loop, or sleeve 74, or other hardware that would otherwise contact the facet joint 10 at issue. Specifically, the buttress 76 is positioned between the cord 60 or sleeve 74 and the facet joint 10, but does not surround the cord 60, as does the sleeves 74 described above. The buttress 76 merely comprises an insert, pad, or other element that generally is positioned between the bone structure of the facet joint 10 and that portion of the cord 60 or sleeve 74 most closely contacting that bone structure. The buttress 76 may comprise a groove or other deformation therein to complement the dimensions of the cord 60 or sleeve 74. In this manner, given the example of a groove formed in the buttress 76, the groove can, but does not necessarily need to, be oriented along the path of the loop between the loop and the facet joint 10 to help position the buttress 76 accurately and securely beneath the loop material. As is noted above in the context of compressible sleeves 74, it is contemplated that the buttress 76 may be configured to provide varying degrees of compressibility to permit variations in facet stabilization. It is further contemplated that the buttress 76 may be configured so as to be filled with a liquid or gas composition. The adding or removing of quantities of the liquid or gas composition allows for the variation of the buttress' degree of flexibility and, thereby, alters the degree of compression about the facet joint 10. This adding or removing of liquid or gas in the buttress 76 may be performed either in an open or a percutaneous surgical procedure through the use of a connection tube and valve.

Furthermore, the fixation device 40 may be configured to enable adjustment of the degree of compression of the cord 60 around the facet joint 10. For example, but not by way of limitation, a length of the primary end of the cord 60 and a length of the secondary end of the cord 60 are wrapped in a fixation device 40 configured as a thumbscrew thereby defining the degree of tension of the cord 60 around the facet joint 10. This thumbscrew, generally, but not necessarily, positioned just beneath the skin surface, is configured to enable the adjustment of the degree of compression applied by the cord 60 around the facet joint 10. As the thumbscrew is turned in one direction, the degree of compression is lessened, while turning the thumbscrew in the opposite direction increases the degree of compression. It is contemplated by the present invention that the lengths of both ends of the cord 60 may pass through a sleeve 74 prior to being wrapped in a thumbscrew. This sleeve 74 may be configured to provide counteraction on the two lengths of cord 60 applying the degree of compression around the facet joint 10. Furthermore, a greater degree of compression may be applied by the cord 60 when the sleeve 74 is pushed toward the facet joint 10. One or more buttresses 76 may be provided as well to provide a greater degree of compression to the facet joint 10. The buttress 76 generally is positioned between the facet joint 10 and the two ends of the cord 60, which may or may not pass through a sleeve 74 prior to being wrapped in the thumbscrew.

The above description relates to one technique of allowing the intra-operative and postoperative adjustment of the degree of compression applied by the facet stabilization hardware to the facet joint 10. It is contemplated that this system, utilizing the loop of the cord 60, sleeves 74, buttresses 76, and any combinations thereof, constitute only one application. The concept of being able to modify the degree of compression is a novel one and it is contemplated that additional devices within the scope of the present invention will allow this novel approach to be achieved.

Yet another embodiment of the present invention, shown in FIG. 6, relates to a method of stabilizing a facet joint through the threading of a cord 60 in a transfacet fashion. It is contemplated by the present invention that the cord 60 may be any other similar device that would serve to stabilize the facet joint 10 under this method. For example, but not by way of limitation, the cord 60 may be, or may be replaced with, a flexible cord, a cable, a bolt, a rod, or any other similar device. This method typically comprises removing corresponding portions of the inferior articular facet 12 and the superior articular facet 14 so as to provide a cord accommodating channel 72 in the inferior articular facet 12, in the superior articular facet 14, and across the interfacet space 18. The method generally further comprises threading a length of a primary end of a cord 60 through the channel and then binding with at least one fixation device 40 the primary end and the secondary end to the inferior 12 and the superior articular facets 14 with a degree of compression sufficient to stabilize the facet joint 10.

For example, but not by way of limitation, as shown in FIG. 6, a flexible cord 60 may be threaded through the channel and then bound at the cord's 60 two ends to the inferior 12 and the superior 14 articular facets with a washer/crimp assembly. The washer is held in compression by the crimp that engages an end of the cord 60. It is contemplated by the present invention using a washer/crimp assembly fixation device that a sleeve 74, or other similar device, may be positioned between the washer and the crimp of a washer/crimp assembly fixation device. This sleeve 74, determined by its degree of compressibility, is configured to allow for greater flexion and extension of the stabilized facet joint 10. Regarding FIG. 6, where two different embodiments of a transfacet cord 60 forming a loop configuration and one embodiment of a transfacet cord 60 not formed in a loop are illustrated, it is noted that the facet stabilization schemes illustrated therein are presented together, along the same spinal column, merely for the convenience of illustration.

The fixation device 40 provided with this method typically is configured as a washer/nut assembly, or other similar device or assembly, wherein the fixation device 40, or an aspect of the fixation device 40, comprises a broad surface. The broad surface may be configured in a variety of shapes, sizes, and contours so as to proportionally fit variously sized facets. This broad surface is positioned about a portion, or portions, of the facet joint 10 and thereby dispenses the compressive force over a wide area of the inferior articular facet 12 and/or superior articular facet 14 as the fixation device 40 engages the cord 60. The degree of compression provided by the broad surface of the fixation device 40 allows for the immediate stabilization of the facet joint 10. This immediate stabilization may be particularly useful in stabilizing osteoporotic, or otherwise compromised, bone.

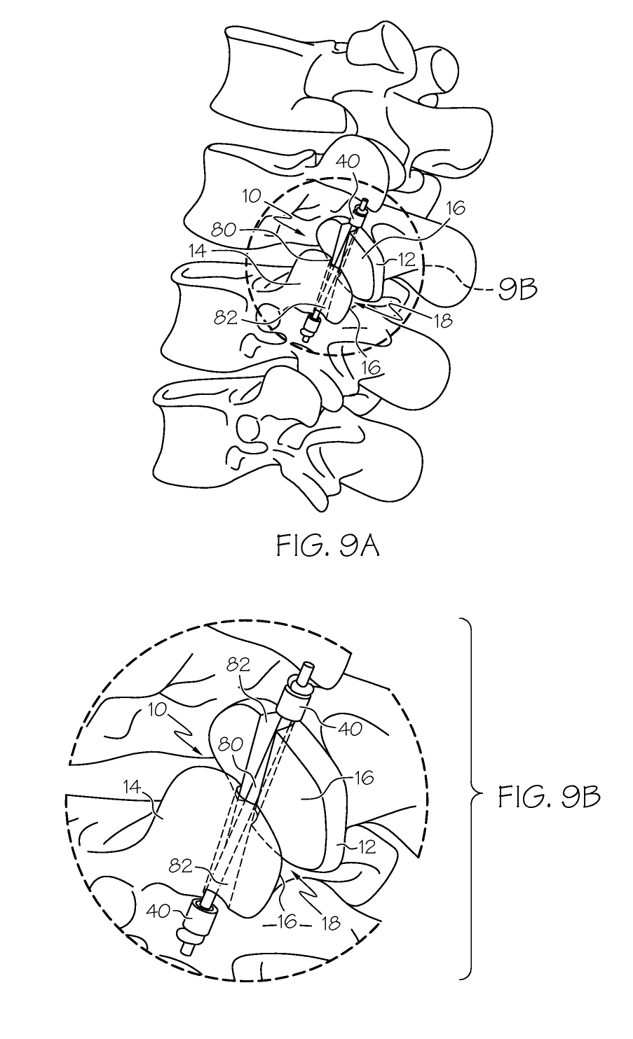

Another embodiment of the present invention, shown in FIGS. 9A and 9B, relates to the stabilization of a facet joint 10 through the use of facet stabilization hardware 80 inserted through the interfacet space 18. This method comprises orienting a bone removal tool along a primary axis in the interfacet space 18 substantially parallel to the opposing faces 16 of the inferior 12 and superior 14 articular facets. The orienting of the bone removal tool may be aided through the use of a guide wire that is inserted into the interfacet space 18. The bone removal tool generally is a drill bit, but may be any other similar bone removing device. In one embodiment, the drill bit comprises a centered hole in its longitudinal axis, through which the drill bit is slid over a guide wire and into the interfacet space 18.

Corresponding portions of the inferior articular facet 12 and the superior articular facet 14 are removed by tilting the bone removal tool with respect to the primary axis about a pivot point along the primary axis. This bone removal tool defines an operative removal surface extending at least partially beyond the bounds of the interfacet space 18 to an extent sufficient to provide a hardware accommodating channel 82 through the inferior articular facet 12, the superior articular facet 14, or both. In the illustrated embodiment, the hardware accommodating channel 82 extends through the inferior articular facet 12 and the superior articular facet 14 along projection defined about a pivot point within the interfacet space 18, near the centroid of the facet joint 10.

The method illustrated in FIGS. 9A and 9B further comprises positioning facet stabilization hardware 80 in the hardware accommodating channel 82 such that one or both ends of the hardware 80 are exposed from cortices of the inferior articular facet 12 and the superior articular facet 14. This facet stabilization hardware 80 is configured to provide a degree of compression sufficient to stabilize the facet joint 10. Such hardware 80 typically, but not necessarily, is a cord 60 bound at its ends to the inferior 12 and superior 14 articular facets by at least one fixation device 40.

For purposes of describing all of the methods of the present invention, it is contemplated that these methods may be achieved through either an open or a closed, minimally invasive, surgical procedure. Furthermore, while they are demonstrated on a lumbar spine, they may be implemented on any portion of the entire spine (lumbar, thoracic, or cervical). Application of any of the herein described methods of the present invention to stabilize a facet joint 10 may be substantially aided by utilizing a percutaneous device that would precisely align any bone removing devices, or other components, used in these methods. Such alignment may be attained by positioning coplanar facet locating caps, which are integral to, and extend from, the percutaneous device, on both the inferior articular facet 12 and the superior articular facet 14. This alignment would ensure the creating of corresponding channels in the inferior 12 and superior 14 articular facets and would provide protection from the over insertion of a bone removing device, or other device, through and past the facets and into sensitive areas of the patient's anatomy. Providing such a device would greatly aid in the closed, minimally invasive, performance of the herein described methods of the present invention.

Furthermore, in describing and defining the all of the methods of the present invention, it is noted that a stabilized facet joint 10 may be completely immobilized or may permit some limited degree of relative movement between the inferior articular facet 12 and the superior articular facet 14. Therefore, the stabilization of the facet joint 10 may be either static or dynamic with the level of stabilization dependent upon the configuration of the facet stabilization hardware 80 in use and the preferences of those practicing the present invention.

It is contemplated by the present invention that all of the above described components utilized in the all of the above described embodiments of methods of stabilizing a facet joint, may be fabricated from a variety of suitable materials including, but not limited to, surgical grade stainless steel, titanium, other metals and metal alloys, synthetic materials, carbon, graphite, combinations thereof, or any other suitable surgical material. In addition, these components may be provided with diagnostically-opaque features. For purposes of the present invention, the term “diagnostically-opaque” means that these components may be diagnostically recognized through advanced diagnostic imaging procedures, such as, but not limited to, X-ray, CAT-scan, fluoroscopy, magnetic resonance imaging (MRI). With the components utilized in stabilizing a facet joint being internally implanted into the patient, there is a need for diagnostic recognition of these components to ensure their structural integrity without the need for a surgical procedure. Therefore, the diagnostically-opaque features allow for the periodic non-invasive monitoring of the structural integrity of the components utilized in stabilizing a facet joint. It is contemplated that the components utilized in stabilizing a facet joint 10, may be provided with diagnostically-opaque features in a variety of ways. For example, and not by way of limitation, diagnostically-opaque material may be provided in discrete elements, or dispersed within the material forming the components or provided in any other manner that would permit the structural state of the components to be monitored periodically over time. In addition, specific teachings regarding the material forming any of these components may be gleaned from existing or yet to be developed technology related to such surgical components.

In many instances, the axis of rotation associated with each vertebral structure during flexion and extension of the spine is most closely aligned with the facet joint, as opposed to the vertebral disc or spinous process. Indeed, it is often the case that the vertebral disc and spinous process are on opposite sides of the axis of rotation defined by a flexing and extending spine. Accordingly, the methods of stabilizing a facet joint described herein can be applied to a position closely approximating the location of this axis of rotation for the specific vertebral levels at issue. In this manner, dynamic stabilization can be enhanced when so desired.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “device” is utilized herein to represent a combination of components and individual components, regardless of whether the components are combined with other components.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1-11. (canceled)

12. A method of stabilizing a facet joint comprising an inferior articular facet of the superior level, a superior articular facet of the inferior level, and an interfacet space defined between said inferior and superior articular facets, said method comprising:
providing a fusion cage configured to maintain a substantial degree of structural integrity when subject to flexion and extension in said facet joint; and
inserting said fusion cage into said interfacet space such that said fusion cage engages said inferior articular facet and said superior articular facet.

13. The method of claim 12, wherein said method further comprises removing corresponding portions of said inferior articular facet and said superior articular facet that define said interfacet space so as to provide an expanded interfacet space.

14. The method of claim 12, wherein said fusion cage is configured with a degree of porosity sufficient to permit diffusion of biological substances into or from an interior space of said fusion cage.

15. The method of claim 14, wherein said biological substances comprise bone or bone growth enhancing materials.

16. The method of claim 12, wherein an exterior surface of said fusion cage is provided in a fastening configuration so as to securely engage with said inferior articular facet and said superior articular facet.

17. The method of claim 12, wherein said method further comprises engaging at least one fixation device to said fusion cage such that said fixation device is securely positioned about said facet joint with a degree of compression sufficient to stabilize said facet joint.

18. The method of claim 14, wherein said interior space of said fusion cage is provided in a fastening configuration.

19. The method of claim 18, wherein said fixation device is configured so as to correspond with said fastening configuration of said interior space.

20. The method of claim 18, wherein said fixation device is configured to compress said facet joint as said fixation device is engaged with said interior space of said fusion cage.

21. The method of claim 22, wherein said method further comprises stabilizing said facet joint by coupling with coupling mechanisms said facet joint to another facet joint.

22. The method of claim 21, wherein said coupling mechanisms are coupled to aspects of the spine other than said facet joints.

23. The method of claim 21, wherein said fusion cage, said fixation device, and said coupling mechanisms comprise diagnostically-opaque material.

24. A method of stabilizing a facet joint comprising an inferior articular facet of the superior level, a superior articular facet of the inferior level, and an interfacet space defined between said inferior and superior articular facets, said method comprising:
removing corresponding portions of said inferior articular facet and said superior articular facet so as to provide a cage accommodating channel in said inferior articular facet, in said superior articular facet, and across said interfacet space; and
providing a fusion cage configured to maintain a substantial degree of structural integrity when subject to flexion and extension in said facet joint; and
inserting said fusion cage into said channel such that said fusion cage engages said inferior articular facet and said superior articular facet and passes across said interfacet space.

25. The method of claim 24, wherein said fusion cage is configured with a threaded leading edge.
